(19) United States
(12) Patent Application Publication
(10) Pub. No.: US 2020/0276344 A1
(43) Pub. Date: Sep. 3, 2020

(54) NEBULIZATION NOZZLE FOR AN APPARATUS FOR SURFACE DISINFECTION BY AIR

(71) Applicant: AIRINSPACE, ELANCOURT (FR)

(72) Inventors: Pierre DE LINAGE, BOULOGNE-BILLANCOURT (FR); Nicolas KLACZYNSKI, JOUARS-PONTCHARTRAIN (FR)

(21) Appl. No.: 16/645,557

(22) PCT Filed: Sep. 7, 2018
(86) PCT No.: PCT/EP2018/000429

§ 371 Date: Mar. 9, 2020

(30) Foreign Application Priority Data
Sep. 8, 2017 (FR) 1770945

(51) Int. Cl. A61L 2/22; B05B 7/10

(52) U.S. Cl. CPC A61L 2/22; A61L 9/14; B05B 7/10

(57) ABSTRACT

A nebulization nozzle for an apparatus for surface disinfection by air, substantially rotating about an axis, including a substantially cylindrical chamber, the axis of which coincides with the axis of the nozzle, a pipe opening into the bottom of the chamber, preferably in the center of the bottom, capable of supplying a treatment product, an air inlet circumventing the periphery of the chamber, vanes arranged between the air inlet and the chamber, and a substantially conical diffuser opening outwards, the axis of which is aligned with the axis of the nozzle and attached to the chamber on the side opposite the bottom, wherein the vanes are curved.

The present patent application claims the priority of French patent application FR 17/70945 filed on 8 Sep. 2017, which is incorporated herein by reference.

### BACKGROUND OF THE INVENTION

### Field of the Invention

The present invention relates to the field of disinfection of an environment by air. It is known in this field to nebulize a treatment product, such as a biocidal product, by mixing it with propellant air in order to diffuse it in fine droplets into the environment, in order to treat the surfaces thereof.

### Description of the Related Art

It is known for this purpose to use an apparatus 20, such as that illustrated in FIG. 1, comprising a tank 21 of treatment product, a means of transport, comprising for example a pump 22, and a pipe 25 (or 4) for this product to a nozzle 24 (or 1), and a motorized fan 23 capable of blowing air to said nozzle 24. The function of the nozzle 24 is to nebulize the treatment product, mix it with the propellant air, and allow the air/product mixture to diffuse into the environment to be treated.

As illustrated in FIGS. 2 and 3, such a nebulization nozzle 1 is a part substantially rotating about an axis 2, comprising a substantially cylindrical chamber 3, the axis of which coincides with the axis 2. The treatment product enters the nozzle 1 through a pipe 4. This pipe opens into the bottom 5 of the chamber 3, preferably in the center of said bottom 5. The nozzle 1 further comprises an air inlet 6. This air inlet 6 receives air from the motorized fan. It is circular around the periphery of the chamber 3 so as to allow air to enter the chamber 3. Vanes 7 are arranged between the air inlet 6 and the chamber 3 in order to guide the airflow. The nozzle 1 further comprises a substantially conical diffuser 8 opening outwards, the axis of which is aligned with the axis 2 of the nozzle 1. This diffuser 8 is attached to the chamber 3 on the side opposite the bottom 5. According to the prior art, as more particularly illustrated in FIGS. 2 and 3, the vanes 7 are flat and substantially radial in relation to the axis 2.

Such a nozzle 1 allows the treatment product to be nebulized and diffused into an environment. However, a particle-size analysis of the outflow from such a nozzle 1 shows a very wide and irregular droplet size spectrum, as shown in FIG. 4, which is detrimental in terms of disinfection effectiveness.

Therefore, the apparatus 20 and/or nozzle 1 should be improved in order to homogenize the droplet size, reduce the droplet size spectrum, in order to improve disinfection effectiveness.

### SUMMARY OF THE INVENTION

The present invention resolves these various disadvantages and provides, according to a first object, a nebulization nozzle for an apparatus for surface disinfection by air, substantially rotating about an axis, comprising a substantially cylindrical chamber, the axis of which coincides with the axis of the nozzle, a pipe opening into the bottom of the chamber, preferably in the center of said bottom, capable of supplying a treatment product, an air inlet circumventing the periphery of the chamber, vanes arranged between the air inlet and the chamber, and a substantially conical diffuser opening outwards, the axis of which is aligned with the axis of the nozzle and attached to the chamber on the side opposite the bottom, wherein the vanes are curved.

A second object relates to an apparatus for surface disinfection by air comprising a tank of treatment product, a means of transport, comprising for example a pump, and a pipe for this product to a nozzle, and a motorized fan capable of blowing air to said nozzle, which nozzle is as described above.

A third and last object of the invention relates to a method for surface disinfection by nebulizing a treatment product.

### BRIEF DESCRIPTION OF THE DRAWINGS

FIG. 1 shows an apparatus for surface disinfection by air.

FIG. 2 shows a cross-sectional side view of a nozzle according to the prior art.

FIG. 3 shows a front of a nozzle according to the prior art.

FIG. 4, shows the particle-size analysis of the jet obtained by means of the nozzle in FIGS. 2 and 3.

FIG. 5 shows a cross-sectional side view of a nozzle according to the invention.

FIG. 6 shows a front view of a nozzle according to the invention.

FIG. 7 shows the particle-size analysis of the jet obtained by means of the nozzle in FIGS. 5 and 6.

FIG. 8 shows a cross-sectional side view of another nozzle according to the invention.

FIG. 9 shows a front view of this another nozzle according to the invention.

FIG. 10 shows a cross-sectional side view of still another nozzle according to the invention.

FIG. 11 shows a front view of this still another nozzle according to the invention.

### DESCRIPTION OF THE PREFERRED EMBODIMENTS

A first object of the invention is directed to a nebulization nozzle for an apparatus for surface disinfection by air, substantially rotating about an axis, comprising a substantially cylindrical chamber, the axis of which coincides with the axis of the nozzle, a pipe opening into the bottom of the chamber, preferably in the center of said bottom, capable of supplying a treatment product, an air inlet circumventing the periphery of the chamber, vanes arranged between the air inlet and the chamber, and a substantially conical diffuser opening outwards, the axis of which is aligned with the axis of the nozzle and attached to the chamber on the side opposite the bottom, wherein the vanes are curved.

According to another feature, the vanes are parallel to the axis of the nozzle and equally angularly distributed around the axis of the nozzle.

According to another feature, the concavity of the curvature of the vanes is directed inwardly of the nozzle.

According to another feature, the vanes have a spiral shape initiated on a circle centered on the axis of the nozzle.

According to another feature, the spiral is of the logarithmic type.

According to another feature, the number of vanes is between 3 and 10, and preferably equal to 5.

According to another feature, the diffuser has a rounded fillet on its internal trailing edge.

According to another feature, the diffuser comprises, away from the chamber, a first cone, a second cone with a larger diameter, and a step between the two cones.

According to another feature, the step has a rounded fillet.

A second object relates to an apparatus for surface disinfection by air comprising a tank of treatment product, a means of transport, comprising for example a pump, and a pipe for this product to a nozzle, and a motorized fan capable of blowing air to said nozzle, which nozzle is as described above.

A third and last object of the invention relates to a use of a nozzle as described above for surface disinfection by nebulizing a treatment product.

Other features, details and advantages of the invention will be more clearly apparent from the detailed description given below for illustrative purposes in connection with the drawings, in which:

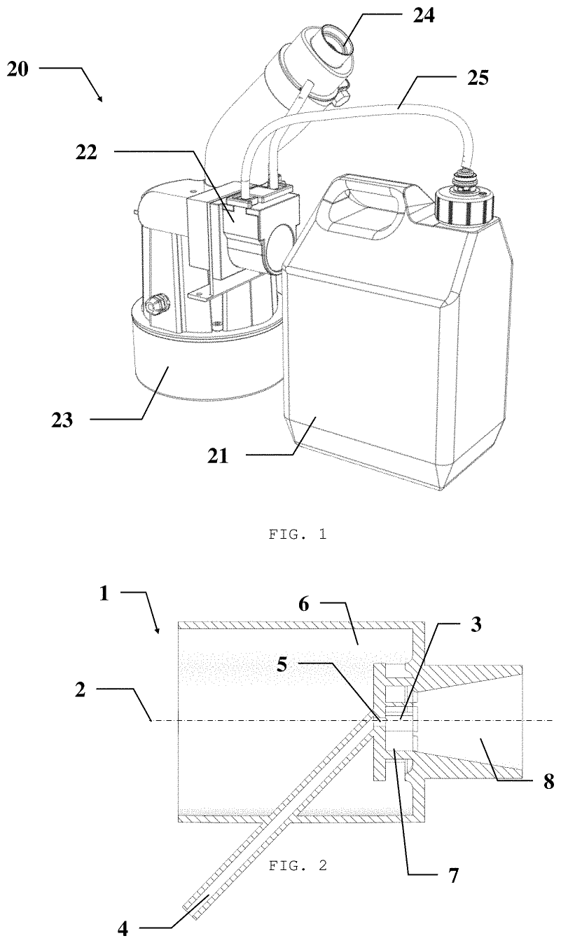

FIG. 1, already described, shows an apparatus for surface disinfection by air,

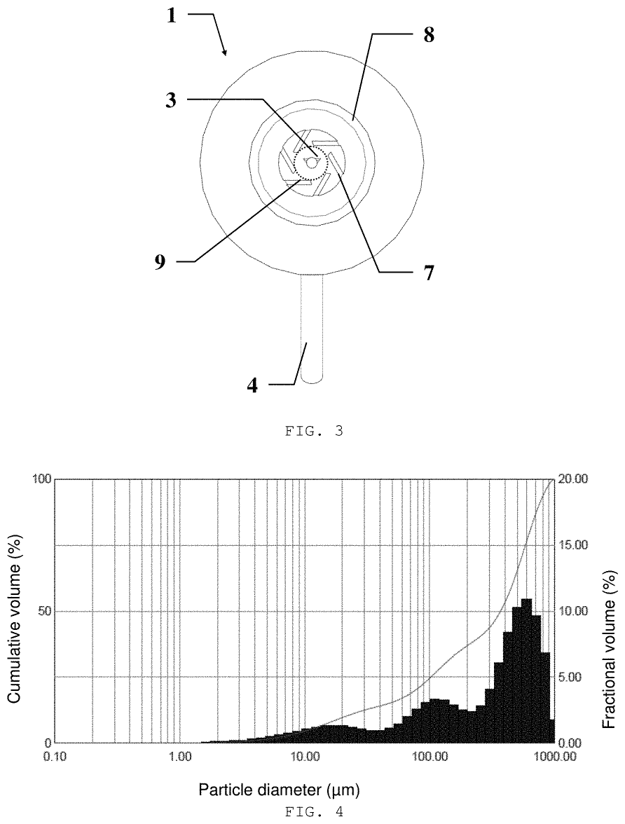

FIGS. 2 and 3, already described, show a nozzle according to the prior art, in a cross-sectional side view and in a front view, respectively,

FIG. 4, already described, shows the particle-size analysis of the jet obtained by means of the nozzle in FIGS. 2 and 3,

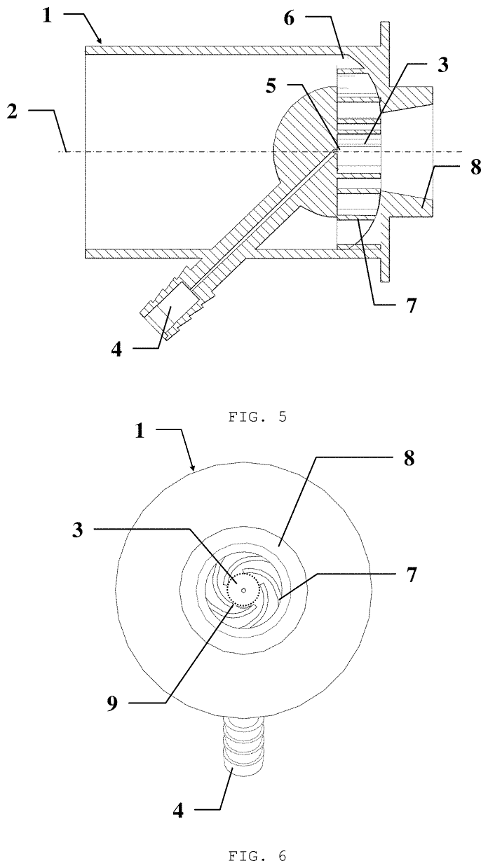

FIGS. 5 and 6 show a nozzle according to a first embodiment of the invention, in a cross-sectional side view and in a front view, respectively,

FIG. 7 shows the particle-size analysis of the jet obtained by means of the nozzle in FIGS. 5 and 6,

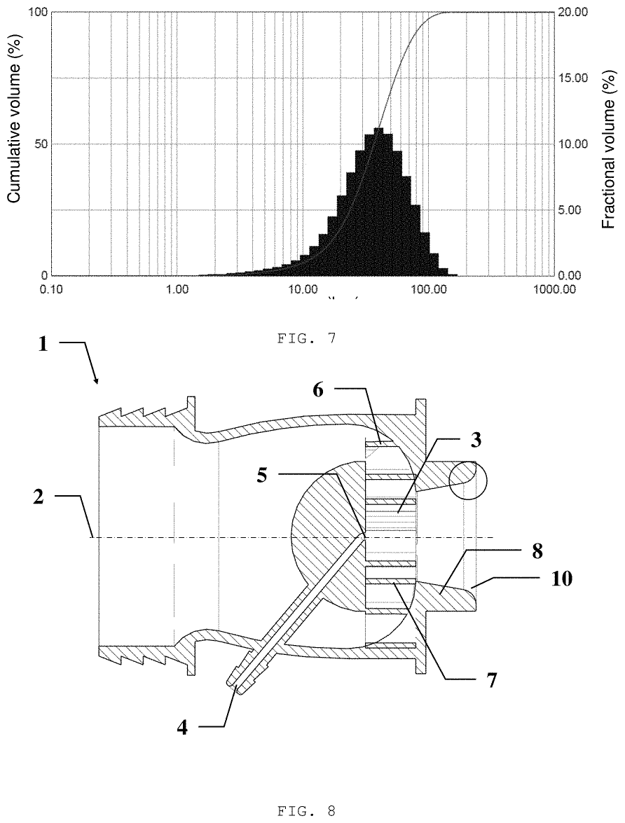

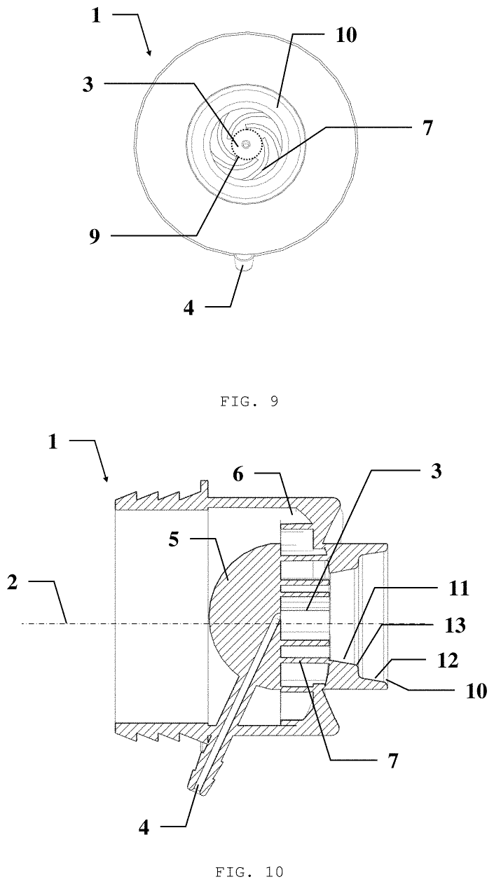

FIGS. 8 and 9 show a nozzle according to another embodiment of the invention, in a cross-sectional side view and in a front view, respectively,

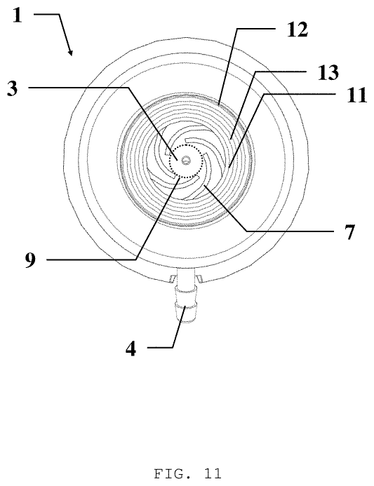

FIGS. 10 and 11 show a nozzle according to yet another embodiment of the invention, in a cross-sectional side view and in a front view, respectively.

According to a first feature of the invention, more particularly illustrated in FIGS. 5 and 6, a nozzle is in all respects identical to a nozzle of the prior art as illustrated in FIGS. 2 and 3. However, it differs and is characterized in that the vanes 7 are curved. This curvature advantageously allows the air entering the chamber 3 to be set in a circular motion in a vortex. This vortex results in a better nebulization of the treatment product which leads to a better homogeneity of the droplets with a less extended spectrum. This is particularly visible in FIG. 7, which shows the particle-size analysis of the jet obtained by means of the nozzle in FIGS. 5 and 6 with curved vanes 7. The particle-size analysis is presented as a spectrum showing the droplet diameter on the abscissa, expressed in microns, and the frequency on the ordinate, expressed as a percentage, for the bar graph, and the cumulative volume on the ordinate, expressed as a percentage, for the continuous curve. It can thus be observed, by comparing the spectrum in FIG. 4, corresponding to a nozzle according to the prior art, with the spectrum shown in FIG. 7, corresponding to a nozzle according to the invention, that the distribution of the droplets has a better regularity, with a substantially Gaussian spectrum and, above all, with an advantageously much smaller range. Such a narrow range spectrum improves the control of the diffusion cone. The distribution of the droplets further has the advantage of a much smaller average diameter, which further promotes nebulization.

According to another feature, the vanes 7 are parallel to the axis 2 of the nozzle 1. Furthermore, they are advantageously equally angularly distributed around the axis 2 of the nozzle 1.

In order to optimize the vortex effect obtained due to the curvature of the vanes 7, this curvature is the same for all vanes 7 and is such that its concavity faces the inside of the nozzle 1.

According to a preferred embodiment, the curve of the vanes 7 has a spiral shape. In order to leave part of the chamber 3 free, the vanes 7 start on a circle 9 centered on the axis 2 of the nozzle 1.

According to an even preferred embodiment, said spiral is of the logarithmic type.

The number of vanes can be any number. According to a preferred embodiment, it is between 3 and 10. According to an even preferred embodiment, it is equal to 5.

The diffuser 8 was seen to have a conical shape. With a standard internal trailing edge of the diffuser 8, a large droplet size accumulation is observed at the end of the diffuser 8. These droplets do not appear on the spectrum because they are stopped before crossing the measuring instrument. However, this phenomenon is detrimental in that it constitutes a waste of treatment product that is not really useful, since it is not diffused.

In order to eliminate this detrimental effect, the diffuser has a fillet 10 on its internal trailing edge. This fillet 10 is preferably rounded, with a concavity facing the outside of the cone.

Such a modification of the diffuser 8 does not significantly change the particle-size spectrum. The nozzle shown in FIGS. 8 and 9 thus retains the advantages of the invention.

According to another feature, more particularly illustrated in FIGS. 10 and 11, the diffuser 8 comprises, away from the chamber 2, a first cone 11, a second cone 12 with a larger diameter, and a step 13 between the two cones 11, 12.

Such an arrangement advantageously allows the length of the cone to be reduced, reduced to the length of the first cone 11, potentially in contact with the diffused flow, and thus reduces friction between the diffused mixture and the diffuser 8. The second cone 12 contributes, advantageously without contact, to protecting the diffused flow.

The step 13 advantageously has, for the same reasons as above, a rounded fillet, similar to the fillet 10.

Such a modification of the diffuser 8 does not significantly change the particle-size spectrum. The nozzle shown in FIGS. 10 and 11 thus retains the advantages of the invention.

1. A nebulization nozzle (1) for an apparatus for surface disinfection by air, substantially rotating about an axis (2), comprising a substantially cylindrical chamber (3), the axis of which coincides with the axis (2) of the nozzle (1), a pipe (4) opening into the bottom (5) of the chamber (3), capable of supplying a treatment product, an air inlet (6) circumventing the periphery of the chamber (3), vanes (7) arranged between the air inlet (6) and the chamber (3), and a substantially conical diffuser (8) opening outwards, the axis of which is aligned with the axis (2) of the nozzle (1) and attached to the chamber (3) on the side opposite the bottom (5), wherein the vanes (7) are curved.

2. The nozzle (1) according to claim 1, wherein the vanes (7) are parallel to the axis (2) of the nozzle (1) and equally angularly distributed around the axis (2) of the nozzle (1).

3. The nozzle (1) according to claim 1, wherein the concavity of the curvature of the vanes (7) is directed inwardly of the nozzle (1).

4. The nozzle (1) according to claim 1, wherein the vanes (7) have a spiral shape initiated on a circle (9) centered on the axis (2) of the nozzle (1).

5. The nozzle (1) according to claim 4, wherein the spiral is of the logarithmic type.

6. The nozzle (1) according to claim 1, wherein the number of vanes is between 3 and 10.

7. The nozzle (1) according to claim 1, wherein the diffuser (8) has a rounded fillet (10) on its internal trailing edge.

8. The nozzle (1) according to claim 1, wherein the diffuser (8) comprises, away from the chamber (2), a first cone (11), a second cone (12) with a larger diameter, and a step (13) between the two cones (11, 12).

9. The nozzle (1) according to claim 8, wherein the step (13) has a rounded fillet.

10. An apparatus for surface disinfection by air comprising a tank 21 of treatment product, a means of transport 22, and a pipe 25 for this product to a nozzle 24 (or 1), and a motorized fan 23 capable of blowing air to said nozzle 24, wherein the nozzle is substantially rotating about an axis (2), comprising a substantially cylindrical chamber (3), the axis of which coincides with the axis (2) of the nozzle (1), a pipe (4) opening into the bottom (5) of the chamber (3), capable of supplying a treatment product, an air inlet (6) circumventing the periphery of the chamber (3), curved vanes (7) arranged between the air inlet (6) and the chamber (3), and a substantially conical diffuser (8) opening outwards, the axis of which is aligned with the axis (2) of the nozzle (1) and attached to the chamber (3) on the side opposite the bottom (5).

11. (canceled)

12. A method for surface disinfection by nebulizing a treatment product comprising the step of nebulizing the treatment product with an apparatus for surface disinfection by air comprising a tank 21 of treatment product, a means of transport 22, and a pipe 25 for this product to a nozzle 24 (or 1), and a motorized fan 23 capable of blowing air to said nozzle 24, wherein the nozzle is substantially rotating about an axis (2), comprising a substantially cylindrical chamber (3), the axis of which coincides with the axis (2) of the nozzle (1), a pipe (4) opening into the bottom (5) of the chamber (3), capable of supplying a treatment product, an air inlet (6) circumventing the periphery of the chamber (3), curved vanes (7) arranged between the air inlet (6) and the chamber (3), and a substantially conical diffuser (8) opening outwards, the axis of which is aligned with the axis (2) of the nozzle (1) and attached to the chamber (3) on the side opposite the bottom (5).

13. The nozzle (1) according to claim 1, wherein the pipe (4) opening into the bottom (5) of the chamber (3) is in the center of said bottom (5).

14. The nozzle (1) according to claim 6, wherein the number of vanes is equal to 5.

15. The nozzle (1) according to claim 2, wherein the concavity of the curvature of the vanes (7) is directed inwardly of the nozzle (1).

16. The nozzle (1) according to claim 2, wherein the number of vanes is between 3 and 10.

17. The nozzle (1) according to claim 3, wherein the number of vanes is between 3 and 10.

18. The nozzle (1) according to claim 4, wherein the number of vanes is between 3 and 10.

19. The nozzle (1) according to claim 5, wherein the number of vanes is between 3 and 10.

20. The nozzle (1) according to claim 6, wherein the diffuser (8) has a rounded fillet (10) on its internal trailing edge.

21. The nozzle (1) according to claim 2, wherein the diffuser (8) comprises, away from the chamber (2), a first cone (11), a second cone (12) with a larger diameter, and a step (13) between the two cones (11, 12).
